(19) United States
(12) Patent Application Publication
(10) Pub. No.: US 2015/0065889 A1
(43) Pub. Date: Mar. 5, 2015

(54) BODILY WORN MULTIPLE OPTICAL SENSORS HEART RATE MEASURING DEVICE AND METHOD

(71) Applicant: Life Beam Technologies Ltd., Tel Aviv (IL)

(72) Inventors: Yosef GANDELMAN, Ashdod (IL); Zvi Orron, Tel Aviv (IL); Yoav Aminov, Tel Aviv (IL); Omri Yoffe, Tel Aviv (IL); Roy Rozenman, Tel Aviv (IL); Jonathan Aprasoff, Herzelia (IL)

(73) Assignee: Life Beam Technologies Ltd., Tel Aviv (IL)

(21) Appl. No.: 14/016,161

(22) Filed: Sep. 2, 2013

(51) Int. Cl. A61B 5/024

(52) U.S. Cl. CPC A61B 5/02427
USPC 600/479

(57) ABSTRACT

A Photoplethysmography-based sensor for measuring heart rate is provided herein. The sensor may include a first light source and a second light source configured to illuminate a body tissue by a first light and a second light respectively; and a first and a second light detectors, each configured to detect light comprising portions of said first light and of said second light, transferred through the body tissue; and a processor with an analog measurement part configured to: receive light intensity readings of at least a portion of light as sensed by each one of both sensors and coming from each one of both sources; and calculate a measure of tissue absorption based on ratios of light portions transmitted by each one of both sources and measured by each one of both detectors.

### FIELD OF THE INVENTION

The present invention relates generally to the field of bodily worn heart rate sensors, and in particular, to such sensors that are based on Photoplethysmography.

### BACKGROUND OF THE INVENTION

Prior to a short discussion of the related art being set forth, it may be helpful to set forth definitions of certain terms that will be used hereinafter.

The term “light source” as used herein may include any component capable of emitting light in the desirable intensity and wavelength, such as a light emitting diode (LED) and the light detector may include any component capable of detecting and measuring the light emitted by the light source, such as a photodiode or phototransistor. Typically, the desirable wavelength of the light source would be within the range of 350-1100 nm.

The term “Photoplethysmography” or PPG as used herein is defined as the use of light traces transmitted through organ tissues in order to analyze physiologic parameters of the organ.

The term “Reflectance Photoplethysmography” or “Reflectance PPG” as used herein is defined as PPG based on measurement of the intensity of light passed through the tissue and reflected back to the same side of the tissue as the light source.

Photoplethysmography is known in the art to be used in measuring heart rate. Heart rate may be detected by analyzing the transmitted light in transmittance PPG or the reflected light in reflectance PPG. Changes of the blood volume in the tissue modify the absorption, reflection or scattering of the light, so the measured reflected or transmitted light varies with the heart cycle. Thus, heart rate may be derived from the measured reflected or transmitted light by means of signal analysis.

The penetration depth of light in biological tissues is typically limited. Therefore, transmittance PPG is typically designed to operate at relatively thin parts of the human body such as the fingertip or the ear lobe. This drawback limits the application of transmittance PPG for heart rate measurements for sport activities. Reflected PPG measurement is not limited in this way and theoretically can be taken at any skin surface at any part of the body.

### BRIEF SUMMARY OF EMBODIMENTS OF THE INVENTION

The present invention, in embodiments thereof, addresses the sensitivity of PPG sensors to movements which may cause undesired noise and inaccurate heart rate measurement. Embodiments of the present invention provide a multi sensor approach that together with a validation process that takes into account the ratio of the incoming signals provides a far more robust PPG sensor for heart rate measurement purposes than PPG based sensors that are currently available.

According to some embodiments, an accelerometer may be further used herein for enhancing the quality and the correctness of the heart rate measuring of the aforementioned heart rate measuring device. The use of an accelerometer may be advantageous in at least three of the following manners: to measure degree of activity of the person wearing the sensing device, to detect a rate of change in that activity and to derive a transfer function of the person wearing the heart rate measuring device. A processor may then use the data collected by the accelerometer and correct the optical measurement accordingly.

According to some embodiments of the present invention, a Photoplethysmography-based sensor for measuring heart rate is provided herein. The sensor takes advantage of two or more light sources and two or more light detectors, wherein a processor analyzed the cross measurements and ratios between the different light and the corresponding reflections. More specifically, the sensor may include a first light source and a second light source configured to illuminate a body tissue by a first light and a second light respectively; and a first and a second light detectors, each configured to detect light comprising portions of said first light and of said second light, transferred through the body tissue; and a processor with an analog measurement part configured to: receive readings of any combination of light intensity as sensed by both sensors and coming from both sources; and calculate a measure of tissue absorption based on ratios of light portions transmitted by each one of both sources and measured by each one of both detectors

These additional, and/or other aspects and/or advantages of the present invention are set forth in the detailed description which follows.

### BRIEF DESCRIPTION OF THE DRAWINGS

For a better understanding of the invention and in order to show how it may be implemented, references are made, purely by way of example, to the accompanying drawings in which like numerals designate corresponding elements or sections. In the accompanying drawings:

FIG. 1 is a schematic illustration of a heart rate sensing device according to some embodiments of the present invention;

FIG. 2 is a schematic block diagram of a system for receiving, processing and presenting to a user heart rate readings based on signals received from heart rate sensing device according to some embodiments of the present invention;

FIGS. 3A and 3B are schematic illustrations of an attachment unit for attaching sensing device to an examined tissue in schematic partial section view and in schematic partial isometric view, respectively, according to some embodiments of the present invention;

FIG. 4 is a schematic illustration of a helmet system comprising heart rate sensor and processing unit according to some embodiments of the present invention;

FIG. 5 is a schematic illustration of a sunglass system for providing heart rate sensor and signals processing and information display management unit according to some embodiments of the present invention;

FIG. 6 is a schematic illustration of a swimming goggles system for providing heart rate sensor and signals processing and information display management unit according to some embodiments of the present invention;

FIG. 7 is a schematic illustration of a heart rate measurement and display system according to some embodiments of the present invention; and

FIG. 8 is a schematic illustration of a heart rate measurement and display system according to some embodiments of the present invention.

The drawings together with the following detailed description make the embodiments of the invention apparent to those skilled in the art.

### DETAILED DESCRIPTION OF EMBODIMENTS OF THE INVENTION

With specific reference now to the drawings in detail, it is stressed that the particulars shown are for the purpose of example and solely for discussing the preferred embodiments of the present invention, and are presented in the cause of providing what is believed to be the most useful and readily understood description of the principles and conceptual aspects of the invention. In this regard, no attempt is made to show structural details of the invention in more detail than is necessary for a fundamental understanding of the invention. The description taken with the drawings makes apparent to those skilled in the art how the several forms of the invention may be embodied in practice.

Before explaining the embodiments of the invention in detail, it is to be understood that the invention is not limited in its application to the details of construction and the arrangement of the components set forth in the following descriptions or illustrated in the drawings. The invention is applicable to other embodiments and may be practiced or carried out in various ways. Also, it is to be understood that the phraseology and terminology employed herein is for the purpose of description and should not be regarded as limiting.

Heart rate measurement devices using reflected PPG are known in the art. A measuring device using reflected PPG as known in the art is usually based on the measurement of the intensity of light passing through the skin of a living tissue from one light source. Current devices do not present a configuration of multiple light sources coupled with multiple sensors for heart rate measurement.

A severe disadvantage of using only one light source and only one light detector is the high sensitivity to artifacts stemming from the relative movement of the measurement device with respect to the measured tissue. The intensity of the light I_{PH} transmitted by a light source which is received by the light detector after passing through an inspected material/tissue may be defined by expression (1) set forth below:

I_{PH}(t)=I_{LED}×K_{LEDcpt}×K_{skinL}(t)×K_{PHcpl} (1)

### Wherein:

I_{LED}—light source Intensity;
K_{LEDcpl}—light source optical coupling coefficient to the inspected material/tissue, which determines the intensity attenuation of the light entering into the material/tissue from the light source;
K_{PHcpl}—Light detector optical coupling coefficient to the inspected material/tissue, which determines the intensity attenuation of the light entering into the Light detector from the material/tissue;
K_{skin L}(t)—Absorption value of light passing distance L within an examined tissue, such as skin;
L—Distance from the light source to the light detector;
t—Time.

As may be seen from equation (1), when the light source intensity and optical coupling coefficients are stable, the I_{PH}(t) represents a stable PPG signal that is proportional to K_{skin L}(t), with the proportionality constant being I_{LED}×K_{LEDcpt}×K_{PHcpl}. This is not true if moving artifacts are present.

The optical coupling coefficients of the light source and the light detector are extremely sensitive to the pressure values in the coupling zone and their values change due to the pressure changes (due to moving artifacts influence). The frequency range of these changes usually coincides with, or in close vicinity to, that of the heart rate. The extent of these changes can be greater than the relative changes of the clean PPG signal. Pressure stabilization can improve the PPG measurement quality.

The coupling coefficients values strongly depend on the magnitude of the pressure at the coupling zone. In the case of low pressures, changes caused by the moving artifact lead to strong changes of the coupling coefficients as a result of the presence of air layer in the coupling zone. When the coupling pressure is high, the sensitivity of the coupling coefficients to the moving artifacts is much lower. This may be because of two different reasons. First reason is—the relative change of the coupling coefficients is defined by the relative pressure change. The relative pressure p=P/P₀ change depends on the moving artifact's acceleration a as in expression (2) set forth below:

p=(P₀±m×a)/a₀ (2)

### Wherein:

P₀—nominal pressure value;
m—mass of the moving part.

As follows from (2), the greater the pressure P₀, the less the p change.

A second reason is that preferably there is no air in the coupling zone between the device and the inspected tissue.

Unfortunately, applying high pressure value to the coupling zone is inconvenient to the user. Moreover, too high pressure may severely interrupt with the blood current in the measurement zone or even completely block it, especially for people with low systolic pressure.

A two light detectors sensing scheme, according to some embodiments of the present invention, which may also be considered as a scheme with a reference light detector, is more stable with respect to artifacts influence. This scheme includes two light detectors and one light source. Light emitted from the light source may reach light detector 1 and light detector 2 after passing through an examined tissue, such as a skin

The intensity of the light I_{PH1} received by the light detector 1 is defined by the expression (3) as set forth below:

I_{PH1}(t)=I_{LED}×K_{LEDcpl}×K_{skin L1}(t)×K_{PH1cpl} (3)

The intensity of the light I_{PH2} received by light detector 2 is as set forth below in expression (4):

I_{PH2}(t)=I_{LED}×K_{LEDcpl}×K_{skin L2}(t)×K_{PH2cpl} (4)

### Wherein:

K_{skin L1}, K_{skin L2}—Absorption value of light passing distance L1 and L2, respectively, within an examined tissue, such as skin;
L1, L2—Distances from light source to light detector 1 and 2, respectively;

From (3) and (4) follows expression (5) below:

K_{skin L2}(t)÷K_{skin L1}(t)=(I_{PH2}(t)/I_{PH1}(t))×K_{PH1cpl}(t)/K_{PH2cpl}(t)) (5)

As follows from (5), the two light detector scheme eliminates or substantially reduces the influence of the changes of optical coupling of the light source, previously denoted K_{LEDcpl}. But this scheme does not eliminate influence of changes of the optical coupling of the light detector.

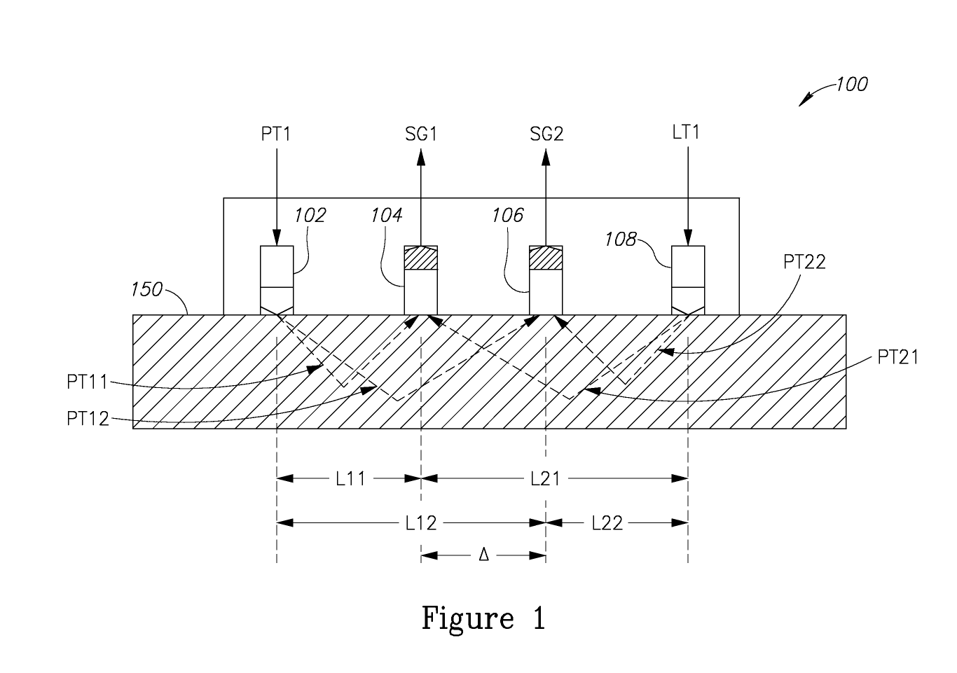

Reference is made to FIG. 1, which is a schematic illustration of heart rate sensing device 100, according to certain embodiments of the present invention. Sensing device 100 may include at least two light sources 102, 108, such as LED type diodes. Sensing device 100 may further include at least two light detectors 104, 106 such as photodiodes. Light sources 102 and 108 may illuminate light in response to light excitation signals LT1 and LT2, respectively. Light sources 102, 108 may be configured to illuminate at two distinguishable lights. For example, light sources 102, 108 may be configured to illuminate at different wavelengths and/or light sources 102, 108 may be configured to alternately illuminate at different time slots. According to some embodiments, light sources 102, 108 may be configured to illuminate light having wavelengths within the range of 350-1100 nm.

Light sources 102, 108 may be arranged distal from each other leaving distance between them for placing light detectors 104, 106 substantially between them, so that a potential path PT12 of a light ray from light source 102 to light detector 106 passes substantially opposite of light detector 104 and a potential path PT21 of a light ray from light source 108 to light detector 104 passes substantially opposite of light detector 106. According to some embodiments of the present invention, light sources 102, 108 and light detectors 104, 106 may be arranged in a row, with light sources 102, 108 at the extremities of the row and light detectors 104, 106 in between. According to some embodiments of the present invention, light source 102 may be placed at a distance of 3-10 mm from light detector 104, light source 108 may be placed at a distance of 3-10 mm from light detector 106, light source 108 may be placed at a distance of 15 mm from light detector 104, and light source 102 may be placed at a distance of 3-10 mm from light detector 106.

Potential path of a light ray from illumination source 102 to light detector 104 will be denoted PT11, and potential path of a light ray from light source 108 to light detector 106 will be denoted PT22. According to some embodiments of the present invention, when sensing device 100 is placed abutting examined article 150, such as a living tissue, light rays along paths PT11, PT12, PT21 and PT22 may pass through, or be reflected from, the outer layers of the tissue of examined article 150 onto light detectors 104, 106. The light received by light detectors 104, 106 may be transmitted by signals SG1, SG2, respectively. Signals SG1, SG2 may be analog or digital signals.

The intensity of the light I_{PH11}(t) detected by light detector 104 from light source 102 along path PT11 is as in expression (6) set forth below:

I_{PH11}(t)=I_{LED1}×K_{LED1cpl}×K_{skin L11}(t)×K_{PH11cpl} (6)

The intensity of the light I_{PH21}(t) detected by light detector 106 from light source 102 along path PT12 is as set forth below in expression (7).

I_{PH21}(t)=I_{LED1}×K_{LED1cpl}×K_{skin L12}(t)×K_{PH21cpl} (7)

The intensity of the light I_{PH12}(t) detected by light detector 104 from light source 108 along path PT21 is as in expression (8) below.

I_{PH12}(t)=I_{LED2}×K_{LED2cpl}×K_{skin L21}(t)×K_{PH12cpl} (8)

The intensity of the light I_{PH22}(t) detected by light detector 106 from light source 108 along path PT22 is as set forth below in expression (9).

I_{PH22}(t)=I_{LED2}×K_{LED2cpl}×K_{skin L22}(t)×K_{PH22cpl} (9)

### Wherein:

I_{LED1}, I_{LED2}—Intensities of light source 102 and light source 108, respectively;
K_{LED1cpl}, K_{LED2cpl}—optical coupling coefficients of light source 102 and light source 108, respectively;
K_{PH11cpl}, K_{PH12cpl}—optical coupling coefficients, when it is illuminated by light source 102 or by light source 108, respectively;
K_{PH21cpl}, K_{PH22cpl}—Light detector 106 optical coupling coefficients, when it is illuminated by light source 102 or by light source 108, respectively;
K_{skin L11}, K_{skin L12}, K_{skin L21}, K_{skin L22}—skin absorption values along paths PT11, PT12, PT21 and PT22, respectively.

According to some embodiments of the present invention, a measure of tissue absorption is calculated based on a ratio of the intensity of light detected by light detector 106 from the light source 102 and the intensity of light detected by the light detector 104 from the light source 102, and a ratio of the intensity of light detected by the light detector 104 from the light source 108 and the intensity of light detected light detector 106 from the light source 108. For example, a measure of tissue absorption is calculated by multiplying the ratio of the intensity of light detected by light detector 106 from the light source 102 and the intensity of light detected by the light detector 104 from the light source 102, by the ratio of the intensity of light detected by the light detector 104 from the light source 108 and the intensity of light detected light detector 106 from the light source 108.

Specifically, from (6)-(9) follows expressions (10) and (11) set forth below:

K_{skin L12}(t)/K_{skin L11}(t)=K_{skin Δ1}=(I_{PH21}(t)/I_{PH11}(t))×(K_{PH11cpl}/K_{PH21cpl}) (10)

K_{skin L21}(t)/K_{skin L22}(t)=K_{skin Δ2}=(I_{PH12}(t)/I_{PH22}(t)×(K_{PH22cpl}/K_{PH12cpl}) (11)

### Wherein:

K_{skin Δ1}, K_{skin Δ2}—skin absorption values for Δ distances, when it is measured in condition of light source 1 (102) or light source 2 (108) illuminating.
   and as in expression (12) below:

$${K}_{skin Δ1} ( t ) \times {K}_{skin Δ2} ( t ) = \frac{{I}_{PH 21} ( t )}{{I}_{PH 11} ( t )} \times \frac{{I}_{PH 12} ( t )}{{I}_{PH 22} ( t )} \times \frac{{K}_{PH 11 cpl}}{{K}_{PH 12 cpl}} \times \frac{{K}_{PH 22 cpl}}{{K}_{PH 21 cpl}} \qquad ( 12 )$$

The propagation of light in the skin is well described by a diffusion theory. The intensity of the passing light has an exponential decay dependence on the distance from the light source, and its exponential form does not depend on the direction of the light propagation. If photo diodes are pressed to the skin and the transmitted light intensity change law is independent of the direction, then

$$\frac{{K}_{PH 11 cpl}}{{K}_{PH 12 cpl}} = \frac{{K}_{PH 22 cpl}}{{K}_{PH 21 cpl}} = 1 \qquad ( 13 )$$

And light detector 104 may measure:

$${K}_{skin Δ1} ( t ) \times {K}_{skin Δ2} ( t ) = \frac{{I}_{PH 21} ( t )}{{I}_{PH 11} ( t )} \times \frac{{I}_{PH 12} ( t )}{{I}_{PH 22} ( t )} \qquad ( 14 )$$

As is seen from equation 14, the expression K_{skin Δ1}(t)×K_{skin Δ2}(t) is independent of all coupling coefficients. In actual measurement conditions, there is some non-uniformity of blood concentration in the skin. Therefore, coupling coefficients do not possess exactly the same values and their ratio is not exactly equivalent to one. But this ratio is much less sensitive to the pressure changes than the light detector coupling coefficient. K_{skin Δ1}(t)×K_{skin Δ2}(t) is a measure of tissue absorption, from which heart rate, and other physiological parameters related to the blood pulse, such as oxygen saturation and arterial stiffness, may be calculated using signal analysis methods.

The low dependence of heart rate sensing device according to the present invention, such as sensing device 100, to changes in the coupling coefficients of the device to the examined article may be used for embedding it in various devices and elements which are worn any way by people active in sport activities, thus eliminating the unpleasant burden of wearing chest strap, as is known in the art.

According to some embodiments of the present invention, sensing device 100 may offer a good ambient light resistance. The system which includes sensing device 100 and signal interface unit 240 may be equipped with one or more current drivers for the light illumination sources, a photocurrent or trans-impedance amplifier(s), an analog to digital convertor(s). The system may further include other units such as: a light illumination current driver(s) controller which changes the current in compliance with the skin absorption and ambient light, an automatic gain control circuit, an ambient light photocurrent compensation controller. Moreover, photo receivers 104 and 106 may include optical filters for an ambient light protection.

According to some embodiments of the present invention, sensing device 100 may offer a mechanical solution to support optics geometry for implementing the aforementioned optical sensing architecture. The sensing device 100 may have an unlimited angle of view of the photo receivers 104 and 106 and so it is very sensitive to small changes of a distance between the sensor and the skin. These changes greatly alter the effective distance Δ between the photo receivers, notably for small Δ values. Therefore, it is desirable that the sensing device 100 may be equipped with elements that may limit the angle of view of the photo receivers 104 and 106. This limiting may be obtained by deepening the photo receivers as shown on FIG. 1 or by an optical guide or by a lens system or by others methods. For stable operation under varying distances between the sensor and the skin it is also desirable to limit the illuminating angles of the light sources. The limitation stabilizes the distances.

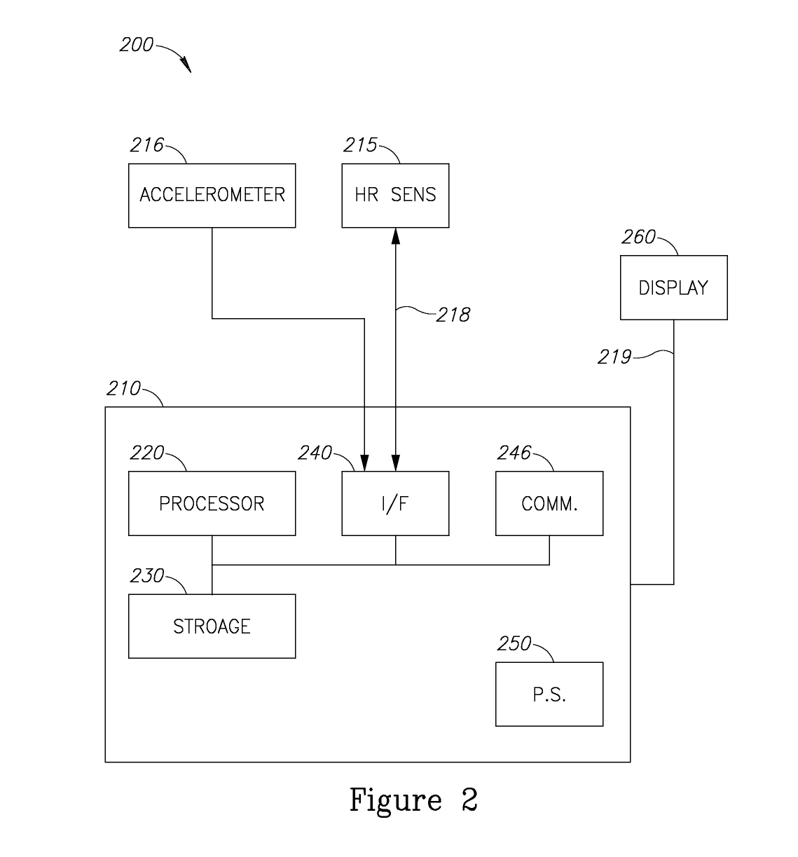

Reference is made now to FIG. 2, which is a schematic block diagram of system 200 for receiving, processing and presenting to a user heart rate readings based on signals received from heart rate sensing device according to some embodiments of the present invention. Heart rate monitoring system 200 may comprise optical sensing unit 215, accelerometer 216, signals processing and display management unit 210 and readings display unit 260. Unit 210 may comprise processing unit 220, memory storage means 230, signal interface (I/F) unit 240, communication unit 246 and power supply unit 250. Sensing unit 215 may be similar or equal to sensing device 100 of FIG. 1. Signals sent to sensing unit 215 to invoke light emitting patterns by light illumination sources, such as light illumination sources 102 and 108 (FIG. 1) and signals received from light detectors such as light detectors 104 and 106 (FIG. 1) may be transmitted between sensing unit 215 and unit 210 via I/F unit 240 and communication channel 218. Communication channel 218 may be embodied via wires or wireless channel. In low power consumption embodiments, as is typical with sport related embodiments, performing channel 218 by wires is preferred, however in some embodiments, where wiring the sensing unit to the processing and presenting unit is impossible, short range wireless solutions may be used, such as Bluetooth (BT) wireless communication. Storage means 230 may be any non-transitory storage means known in the art, such as ROM, PROM. EPROM, EEPROM, DRAM, SDRAM and the like. Storage unit 230 may store data, parameters and program code which when executed by processor unit 220 perform the operations, commands and calculations described throughout this description. It is understood however that processing unit 220 may be implemented as analog circuits and is not limited to digital electronics circuits.

Processor unit 220 may be any suitable processor, processing unit, programmable logical computer (PLC) computer, etc. Typically, the selected processing unit will be as small and light as possible, to allow its embedding in the intended sport related devices and accessories. Processor unit 220 may be adapted to perform program code stored in storage unit 230, to receive signals from sensing unit 215 via I/F unit 240 and to invoke illumination control signals toward sensing unit 215 via I/F unit 240. Unit 210 may be adapted to store user—specific parameters, either entered manually or stored during use and processed to represent the user's specifics in order to provide more accurate readings of the heart rate. Unit 210 may also be adapted to store parameters specific to the sport branch taken by the user and may further be adapted to process the heart rate signals in accordance with these sport specific parameters in order to provide more accurate heart rate readings. Display unit 260 may be any low power, short focus length and light weight display, either containing the display surface as part of it or, according to other embodiments, screening the visual information on a visor surface being an integral part of a hat, glasses, sun glasses or the like. In some embodiments, display unit 260 may be packed together with unit 210 and in other embodiments it may depart from unit 210, for example in order to enable convenient location with respect to the eye of the user. When display unit 260 is located away from unit 210 it may be in active communication with unit 210 via communication channel 219, being wired or wireless channel as may be required.

In order to improve the quality of the heart rate signal picked by a sensor, according to some embodiments of the present invention, such as sensing device 100, proper mechanical installation need to be provided. Most common phenomenon which may induce noise into the heart rate signal picked by a sensor according to some embodiments of the present invention are the movements of the user when in a sportive activity such as walking, running, swimming, riding bicycles and the like, which may cause movements of the sensing device relative to the examined tissue. One of the harshest movements is incurred during running; however, other sportive activities may also induce noticeable relative movements that may deteriorate the quality of the heart rate signal picked by the sensing device. According to some embodiments of the preset invention, movements artifacts may be filtered from the detected signal based on measurements of these movements, for example, by accelerometer 216. Still, there is a need for a mechanical attachment unit that will provide sufficient attachment pressure to sufficiently attach the sensing device to the examined tissue and will allow maximal mechanical detachment of the sensing device from the accessory it is attached to, so as to minimize influence of movements of the accessory relative to the body organ it relates to on the attachment of the sensing device to a tissue of that body organ.

According to some embodiments, accelerometer 216 may be used herein for enhancing the quality and correctness of the heart rate measuring of measuring device 100. The use of an accelerometer may be in at least three manners: to measure degree of activity of the person wearing the sensing device, to detect a rate of change in that activity and to derive a transfer function of the person wearing the heart rate measuring device. Processor unit 210 may then use the data collected by the accelerometer and correct the optical measurement accordingly.

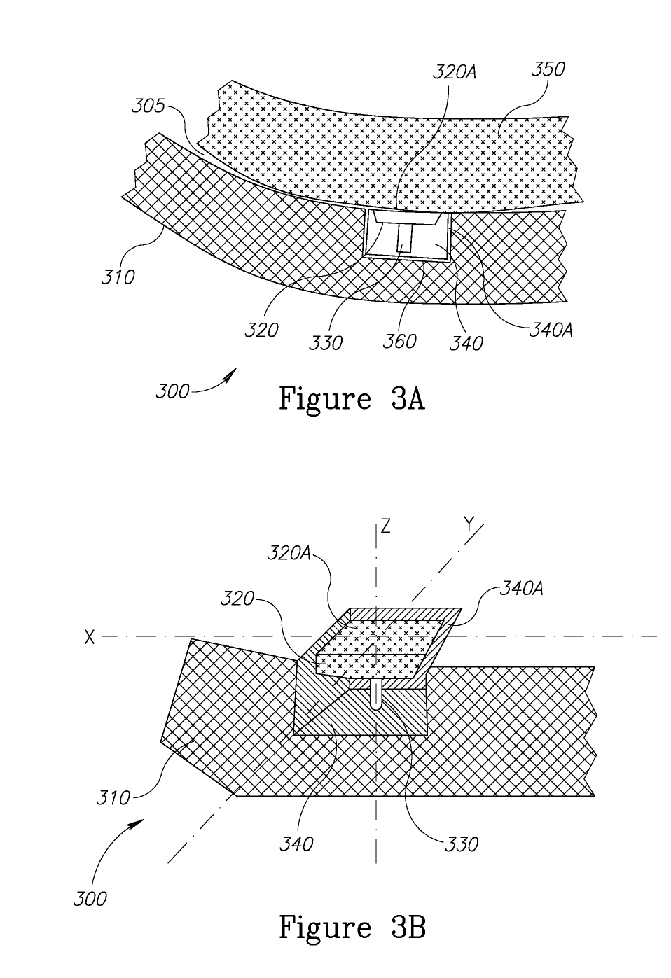

Reference is made to FIGS. 3A and 3B, which schematically illustrate attachment unit 300 for attaching a sensing device to an examined tissue 350 in schematic partial section view and in schematic partial isometric view, respectively, according to some embodiments of the present invention. Attachment unit 300 may comprise a wearable or other attachable accessory 310 in which sensor 320, built and operative according to some embodiments of the present invention, such as sensing device 100 (FIG. 1) may be embedded, for example in recess 340 made in attachable accessory 310 so as to include sensor 320 in it and to enable the face 320A of sensor 320 to attach or about the outer face of examined tissue 350. As seen in FIG. 3B, recess 340 may be made to allow sufficient freedom for movements of sensor 320 within recess 340 along axes X and Y, which define a plane that is parallel to the face 320A of sensor 320. Sensor 320 may be supported by support element 330 which may be formed to provide sufficient attaching force along axis Z substantially perpendicular to face 320A. The attaching force along axis Z provided by support element 330 should be substantially constant or kept within a desired range, for example, support element 330 may be formed to provide pressure of 30-40 mmHg between sensor 320 and the outer face of examined tissue 350. Support element 330 may be flexible enough to allow sensor 340 to conform to the different surfaces of the skin. Support element 330 may concurrently provide sufficient freedom for sensor 340 to move along axes X and Y, to allow for small high-frequency relative movements between sensor 340 and the outer face of examined tissue 350. This arrangement may ensure sufficient attachment of sensor 320 in a direction perpendicular to the adjacent surface of examined tissue 350 while providing mechanical disengagement of sensor 320 from relative movements of attachment accessory 310 with respect to examined tissue 350, thus allowing sensor 320 to provide signal with better S/N ratio. Attachable accessory 310 may include a shell 360 (shown only in FIG. 3A for clarity) to optically isolate the optical sensor from ambient light. Advantageously, the aforementioned structure may guarantee meeting the constant or sufficient pressure requirement applied to the skin by the optical sensor.

Attachable accessory 310 may be any sportive accessory, such as safety helmet, sun glasses, swimming goggles, etc. in each such accessory a respective location for sensor 320 may be selected, to ensure good attachment of sensor 320 to the surface of examined tissue 350. Support element 330 may be implemented in many ways, as is known in the art. For example, support element 330 may be implemented by a thin membrane, made from an elastic fabric or elastomer. Sensor 320 may be attached to the middle of the membrane and the surrounding edge may be connected to attachable accessory 310. According to some embodiments, support element 330 may be implemented by an elastic layer of sponge, made of any applicable material such as silicone or Urethane. The sponge may be placed between sensor 320 and attachable accessory 310. In addition, support element 330 may be implemented by a spring system, connecting sensor 320 to attachable accessory 310.

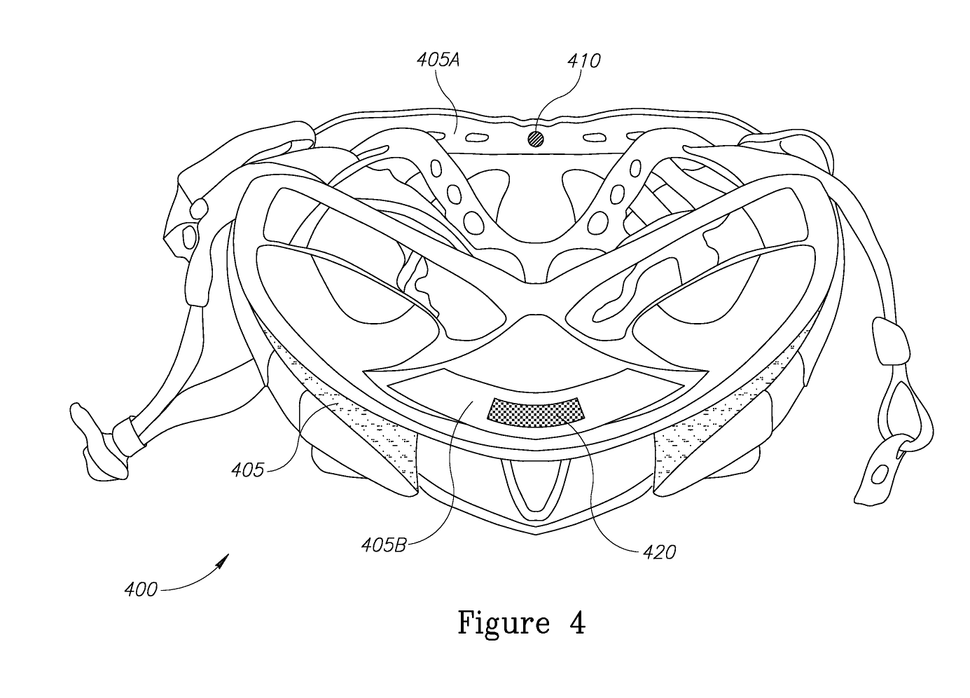

According to some embodiments of the present invention, a heart rate sensing device such as sensing device 100, may be embedded in a safety helmet, such the helmet of a bicycle rider. Reference is made to FIG. 4, which schematically illustrates helmet system 400 comprising sensor 410 according to some embodiments of the present invention. Helmet system 400 may comprise helmet 405, such as helmet used for riding bikes, in which sensor 410 is installed, for example in the forward portion 405A of the head's cushioning belt of helmet 405 so as to enable sensor 410 to be pressed against and abutting the forehead of the bike rider with sufficient sideways movement freedom, as described with respect to drawings 3A and 3B. Signals processing and information display management unit 420, similar to unit 210 of FIG. 2, may be embedded, for example, in the cushioning portion 405B of the scruff. Display of the heart rate and potentially other data may be implemented in several ways, as is discussed herein below.

According to some embodiments of the present invention, sensor 410 and display management unit 420 may be embedded into helmet system 400, or be designed as a standalone heart rate measurement system, adapted to be attachable to standard helmet systems. A standalone heart rate measurement system including sensor 410 and display management unit 420 according to some embodiments of the present invention is advantageous since a user may fit such system to practically any safety helmet. Similarly, the heart rate measurement system may be easily implemented and inserted within a head band.

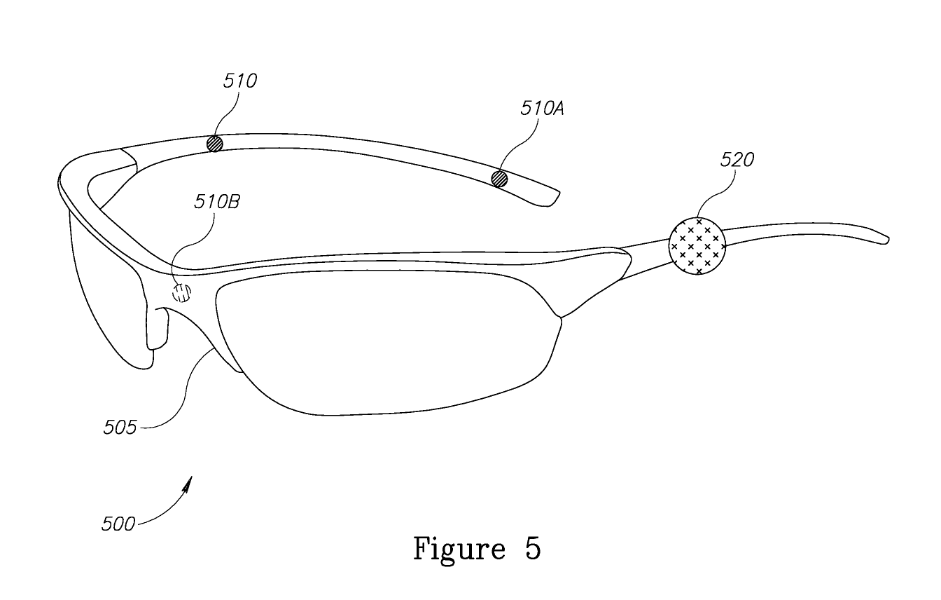

Reference is made now to FIG. 5, which schematically illustrates sunglass system 500 for providing sensor 510, 510A 510B (near the nasal bridge) and signal processing and information display management unit 520 according to some embodiments of the present invention. Sunglass system 500 may include sportive, or other type of sunglass 505 to which heart rate sensor 510, or 510A which are similar to sensor 100 (FIG. 1) may be attached, for example to one of the sunglass's bars so as to place sensor 510, or in a different location sensor 510A, close to the wearer skin and provide a required pressure of sensor 510, 510A to that skin. Signals processing and information display management unit 520 may be located, for example, on the other bar of sunglasses 505. In system 500, sensor 510 or 510A may be connected to unit 520 by wires.

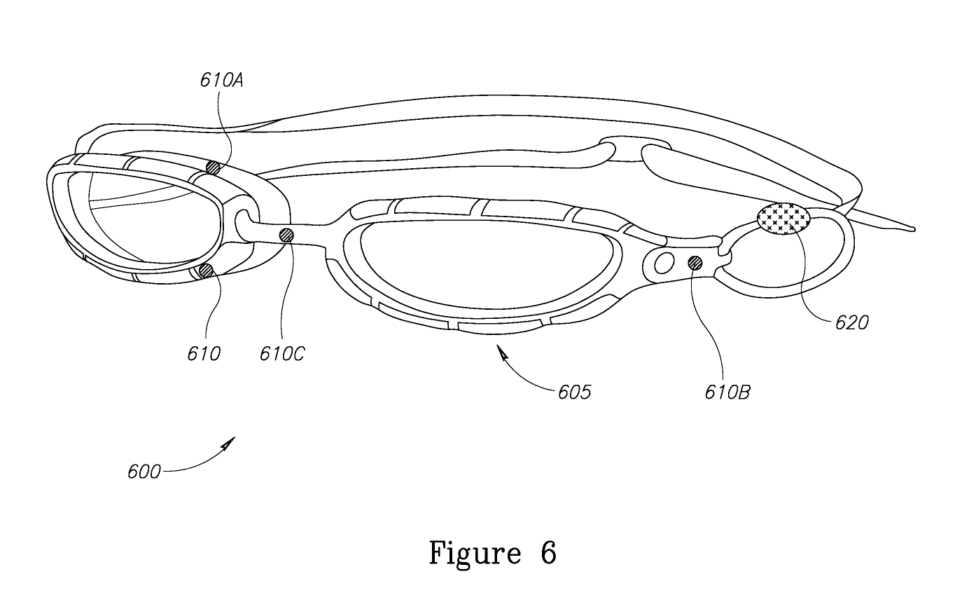

Reference is made now to FIG. 6, which schematically illustrates swimming goggles system 600 for providing sensor 610, 610A and signals processing and information display management unit 620, according to some embodiments of the present invention. Swimming goggles system 600 may comprise sportive or other type of swimming goggles 605 to which heart rate sensors 610 610A, 610B, and 610C which are similar to sensor 100 (FIG. 1) may be attached, for example to one of the goggles' flexible strap so as to place sensor 610, or in a different location sensor 610A 610B, and 610C, close to the wearer's skin and provide a required pressure of sensor 610, 610A 610B, and 610C to that skin. Signals processing and information display management unit 620 may be located, for example, on another portion of the flexible strap of goggles 605. In system 600, sensors 610 or 610A 610B, and 610C may be connected to unit 620 by wires.

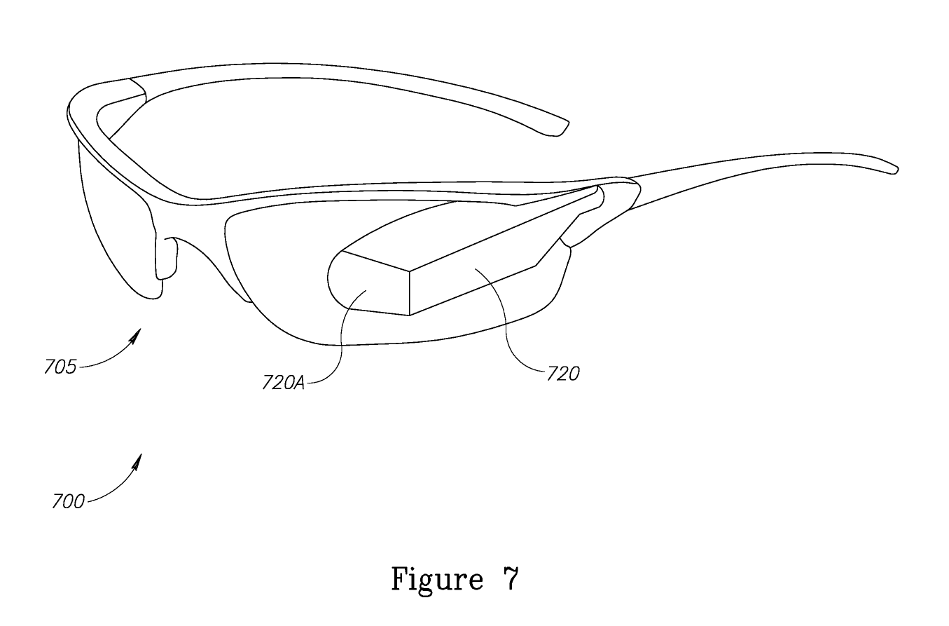

Reference is made now to FIG. 7, which schematically illustrates heart rate measurement and display system 700 according to some embodiments of the present invention. System 700 may comprise sunglasses 705 and heart rate sensor such as sensor 100 and signals processing and information display management unit such as unit 620 both are not shown in this drawing so as to not obscure the drawing. System 700 may further comprise mini-display device 720 attached on one of the glasses of sunglasses 705 placed and oriented so as to enable the respective eye of the sunglasses wearer to conveniently watch images displayed on the inner side of display element 720A.

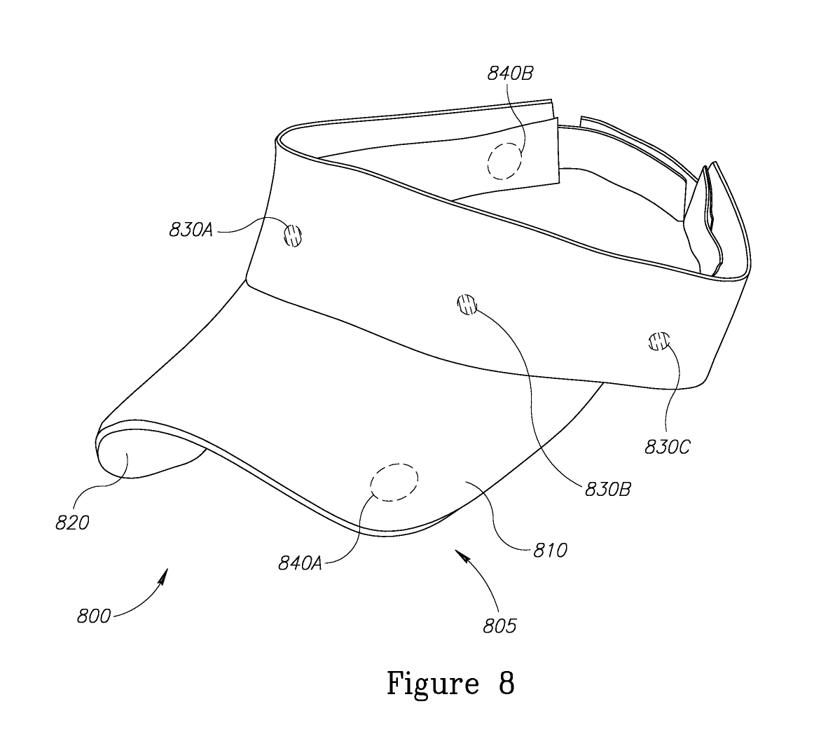

Reference is made now to FIG. 8, which schematically illustrates heart rate measurement and display system 800 according to some embodiments of the present invention. System 800 may comprise eye-shade 805 and heart rate sensor such as sensor 100 and signals processing and information display management unit such as unit 620 both are not shown in this drawing so as to not obscure the drawing. System 800 may further comprise mini-display device 820 attached on one side of the eye-shade 805 placed and oriented so as to enable the respective eye of the eye-shade wearer to conveniently watch images displayed on the inner side of display element 820.

According to some embodiments of the present invention, each of the heart rate measurement and display systems described hereinabove, such as systems 400, 500, 600, 700, 800, may include more than one heart rate sensor such as sensor 100, located, for example, in different parts of the system. Obtaining readings from more than one sensor may enable the processing and information display management units such as unit 420, 520, 620 to produce more accurate results by integrating readings from the more than one sensor, for example by averaging heart rate readings or by disregarding measurements with poor signal quality and relaying of readings with better signal quality. Additionally, each of the heart rate measurement and display systems described hereinabove, such as systems 400, 500, 600, 700, 800, may include an accelerometer to measure accelerations of the user, and use that data to filter movement artifacts from the optical signal.

According to some embodiments, an algorithm that may be implemented in the aforementioned device is presented hereinafter. The aim of the algorithm is to robustly calculate heart-rate from pulse and 3D acceleration signals while the monitored person is non-stationary (e.g., running, cycling, swimming). The algorithm is carried out in real-time and automatically quantifies the quality of the current signal, enhances it by removing motion artifacts, and continuously calculates and tracks the heart-rate.

The algorithm may include five sub-modules as follows: artifacts removal; pulse enhancement; noise cancellation; frequency estimation; and frequency tracking. The artifacts removal module may receive the pulse and acceleration signals in real-time and removes the movement's artifacts from the optical signal using adaptive filters. Then, the pulse enhancement module may emphasize the pulsatile component of the signal and reduce transient noise components. The noise cancellation may automatically identify legal and illegal pulses in a pulse window and pass on only the legal areas for further processing. The frequency estimation module may be applied on windows of data and estimate the dominant frequency in it in several ways (e.g., spectral domain and time domain) and pass the estimated frequencies to the tracking module. The frequency tracking module may be based on a physiological model that allows the heart-rate frequency to change in a realistic way. The predicted frequency may be given back to the frequency estimation module as a feedback in order to enhance the next estimated frequency.

As will be appreciated by one skilled in the art, aspects of the present invention may be embodied as a system, method or an apparatus. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit, “module” or “system.”

The aforementioned flowchart and block diagrams illustrate the architecture, functionality, and operation of possible implementations of systems and methods according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

In the above description, an embodiment is an example or implementation of the inventions. The various appearances of “one embodiment, “an embodiment” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

It is to be understood that the phraseology and terminology employed herein is not to be construed as limiting and are for descriptive purpose only.

The principles and uses of the teachings of the present invention may be better understood with reference to the accompanying description, figures and examples.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is to be understood that where the claims or specification refer to “a” or “an” element, such reference is not be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

The descriptions, examples, methods and materials presented in the claims and the specification are not to be construed as limiting but rather as illustrative only.

Meanings of technical and scientific terms used herein are to be commonly understood as by one of ordinary skill in the art to which the invention belongs, unless otherwise defined.

The present invention may be implemented in the testing or practice with methods and materials equivalent or similar to those described herein.

While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A system comprising:
an optical sensor comprising:
a first and a second light sources configured to illuminate a body tissue by a first light and a second light respectively; and
a first and a second light detectors, each configured to detect light comprising portions of said first light and of said second light, transferred through the body tissue; and
a processor with an analog measurement part configured to:
receive readings of:
intensity of light detected by the first light detector from the first light source,
intensity of light detected by the first light detector from the second light source,
intensity of light detected by the second light detector from the first light source, and
intensity of light detected by the second light detector from the second light source; and
calculate a measure of tissue absorption based on a ratio of the intensity of light detected by the second light detector from the first light source and the intensity of light detected by the first light detector from the first light source, and a ratio of the intensity of light detected by the first light detector from the second light source and the intensity of light detected by the second light detector from the second light source.

2. The system according to claim 1, wherein the processor is further configured to calculate the measure of tissue absorption by multiplying the ratio of the intensity of light detected by the second light detector from the first light source and the intensity of light detected by the first light detector from the first light source, by the ratio of the intensity of light detected by the first light detector from the second light source and the intensity of light detected by the second light detector from the second light source.

3. The system according to claim 1, wherein the first and the second light detectors are placed in between the first and the second light sources.

4. The system according to claim 1, wherein the first and the second light sources and the first and the second light detectors are arranged in a row, wherein the first and the second light sources are placed at the extremities of the row.

5. The system according to claim 1, wherein:
the first light source is placed at a distance of 1.5-10 mm from the first light detector,
the second light source is placed at a distance of 2.5-15 mm from the first light detector,
the first light source is placed at a distance of 2.5-15 mm from the second light detector, and
the second light source is placed at a distance of 1.5-10 mm from the second light detector.

6. The system according to claim 1, wherein each of the first and the second light sources to illuminate light having wavelengths within the range of 350-1100 nm.

7. The system according to claim 8, wherein each of the first and the second light sources is configured to illuminate the tissue at different wavelengths.

8. The system according to claim 1, wherein each of the first and the second light sources to alternately illuminate at different time slots.

9. A helmet comprising the optical sensor of claim 1, wherein the optical sensor is placed in an area of the helmet configured to be abutting a forehead of a user of the helmet.

10. The helmet of claim 11, further comprising a support element configured to generate a pressure of 20-50 mmHg between the optical sensor and the forehead of the user.

11. The helmet of claim 12, wherein the support element is configured to allow freedom for movements of the optical sensor in plane that is parallel to a face of the optical sensor abutting the forehead of the user.

12. The helmet of claim 11, further comprising a shell configured to optically isolate the optical sensor from ambient light.
